(19) United States
(12) Patent Application Publication
(10) Pub. No.: US 2024/0366509 A1
(43) Pub. Date: Nov. 7, 2024

(54) Dry Suspension Granules for Dry Suspension and Preparation Method Thereof

(71) Applicant: LIVZON PHARMACEUTICAL GROUP INC., Zhuhai, Guangdong (CN)

(72) Inventors: Pucheng LI, Zhuhai, Guangdong (CN); Yating MO, Zhuhai, Guangdong (CN); Xiangna ZHANG, Zhuhai, Guangdong (CN); Siwen HU, Zhuhai, Guangdong (CN); Xuemei HOU, Zhuhai, Guangdong (CN); Xiaoman JIANG, Zhuhai, Guangdong (CN); Weishan LIN, Zhuhai, Guangdong (CN); Zhihui HAN, Zhuhai, Guangdong (CN); Yannan CUI, Zhuhai, Guangdong (CN); Caihua CHENG, Zhuhai, Guangdong (CN)

(21) Appl. No.: 18/686,064

(22) PCT Filed: Aug. 25, 2022
(86) PCT No.: PCT/CN2022/114705

§ 371 Date: Feb. 23, 2024

(30) Foreign Application Priority Data
Aug. 26, 2021 (CN) 202110990993.1

(51) Int. Cl. A61K 9/16; A61K 31/4439; C08J 3/00; C08J 3/12; C08L 1/28; C08L 5/00; C08L 5/08; C08L 29/04

(52) U.S. Cl. CPC A61K 9/1652; A61K 9/1635; A61K 9/1694; A61K 31/4439; C08J 3/005; C08J 3/12; C08L 1/28; C08L 1/286; C08L 5/00; C08L 5/08; C08L 29/04; C08J 2301/28; C08J 2305/00; C08J 2305/08; C08J 2329/04; C08J 2401/28; C08J 2405/00; C08J 2405/04; C08J 2405/06; C08J 2405/08; C08J 2439/06

(57) ABSTRACT

Dry suspension granules for dry suspension, including the dry suspension made of the said dry suspension granules, and preparation method thereof are provided. Such-The dry suspension granules contain anionic gel and cationic polymer. The weight ratio of the anionic gel and the cationic polymer is (0.5-50):1.

### PRIORITY

This application claims priority from the application No. 202110990993.1 for the utility patent titled “Dry Suspension Granules for Dry Suspension and Preparation Method and Use Thereof”, which was filed with China National Intellectual Property Administration on Aug. 26, 2021.

### TECHNICAL FIELD

This invention relates to the dry suspension granules for dry suspension, including the dry suspension made of the said dry suspension granules, and preparation method and use thereof.

### BACKGROUND ART

Proton Pump Inhibitors (PPIs) are drugs that can inhibit the H⁺/K⁺-ATPase (also known as Proton Pump or Acid Pump) on the secretory tubule periosteum of human gastric mucosa cells selectively. Since such H⁺/K⁺-ATPase is the ultimate pathway to inhibit the secretion of gastric acid, it is possible to reduce the secretion of gastric acid to a significant extent by inhibiting such H⁺/K⁺-ATPase; therefore, the proton pump inhibitors are usually used to treat the diseases of digestive tract induced or caused by the effect of gastric acid (i.e. the acid-related diseases). Such diseases of digestive tract include gastric and duodenal ulcer, gastroesophageal reflux disease, surgical anastomotic ulcer, and Zollinger-Ellison syndrome. By the mechanism of action, the known PPIs are classified into the non-reversible PPIs and the reversible PPIs (RPPIs). Among them, the non-reversible PPIs are mainly composed of benzimidazole derivatives that can pass through the parietal cell membrane quickly and accumulate in highly acidic secretory tubules. Then, the non-reversible PPIs are protonated and transformed into sulfenamide compounds, which can form the covalently bound disulfide bonds with the sulfhydryl groups on cysteine residues in the a subunits of H⁺/K⁺-ATPase, so that H⁺/K⁺-ATPase is irreversibly inactivated, and its acid-secreting activity is inhibited (Zhang Xuan, “Overview of Patent Technology for Proton Pump Inhibitors-Prazole Drugs”; Patent Literature Research in 2018—Medical & Pharmaceutical, Intellectual Property Publishing House, Beijing, September 2019: p 554-567). So far, the drugs that have been launched in the market in the world include: Omeprazole, Lansoprazole, Pantoprozole, Rabeprazole, Esomeprazole, Ilaprazole, and Delansoprazole, etc.

Similar to other prazole drugs, Ilaprazole is also a kind of acid-unstable compound. However, compared with other existing prazole drugs, Ilaprazole has lower stability. The acid-unstable compounds refer to the substances that are unstable in acidic media but have better stability in neutral and alkaline media. The common feature of such compounds is that they can become biologically effective compounds after rapid degradation/transformation in acid media. In acidic and neutral media, the acid-unstable proton pump inhibitors are sensitive to degradation/transformation; therefore, the acid-unstable proton pump inhibitors shall be prevented from contact with gastric acid when they are used orally, in order not to affect their stability. One of the solutions for this problem is to prepare the enteric pellet preparations by coating the oral preparations of these drugs with enteric materials.

The enteric pellets are usually not administered by means of direct application, but are further made into other forms of dosage such as tablets, capsules or dry suspensions, so as to facilitate the administration. As a kind of powdery or granular material made of insoluble solid drugs and appropriate excipients, the dry suspension can be dispersed into suspension by shaking with water before use. Other liquid dispersion media and additives suitable for oral administration other than water can be added into the dry suspension to improve the taste and other properties. Suspension is a process in which the insoluble solid granules are dispersed almost uniformly in the appropriate liquid dispersion medium by a mechanical method. Due to the high dispersion degree and the large surface free energy, the solid granules in the suspension so formed are more likely to conglomerate, so the suspension is a thermodynamically unstable system. In the suspension, the solid granules are larger than the colloidal particles, and it is prone to sedimentation by gravity, so suspension is also a dynamically unstable system.

Therefore, how to improve the thermodynamic and kinetic properties of the dry suspension, in particular the dry suspension containing pellets when added with dispersion medium (especially water) to make the suspension, such as the time when the stable viscosity level is reached, and the time when the solid granules such as pellets are suspending stably, has become a problem in this field.

### SUMMARY OF THE INVENTION

Surprisingly, the Inventor discovered that by using the combination of anionic gel and cationic polymer (e.g. chitosan and its derivatives) in the dry suspension granules, the resulting suspension can reach a stable viscosity level quickly, and the suspension gel so prepared enables the pellets to suspend stably for a long period of time.

The purpose of this invention is fulfilled with the dry suspension granules containing anionic gel and cationic polymer based on this invention, in particular the said composition of the dry suspension granules enables the pellets to suspend stably in the suspension gel after the suspension/suspension gel is formed. Therefore, with the dry suspension granules based on this invention, it is possible to provide the dry suspension that forms stable suspension gel more quickly, and enable the pellets in the suspension gel formed by such dry suspension to suspend stably for a longer period of time.

Therefore, in one aspect, this invention provides a kind of dry suspension granules for dry suspension, which contains anionic gel and cationic polymer, wherein the weight ratio of the said anionic gel to the said cationic polymer is (0.5-50):1.

According to an embodiment of this invention, the preferable weight ratio of the said anionic gel to the said cationic polymer is (0.8-20):1, more preferably (0.9-10):1, and most preferably (1-3):1.

According to an embodiment of this invention, the said anionic gel is sourced from one, two, or more of Arabic gum, gelatin, alginate (e.g. sodium alginate), pectin, xanthan gum, gellan gum, locust bean gum, guar gum, agar, carrageenan, tamarind gum, konjac gum, cassia bean gum, tragacanth gum, and karaya gum.

According to an embodiment of this invention, the said cationic polymer is sourced from chitosan or its derivatives.

According to an embodiment of this invention, the weight percentage of the said cationic polymer in dry suspension granules is 0.5-5%, preferably 0.6-4%, more preferably 0.8-3.5%, and most preferably 0.9-3.0%.

According to an embodiment of this invention, the dry suspension granules based on this invention also include one, two or more of adhesive, disintegrating agent, diluent, and pH regulator.

According to an embodiment of this invention, the said adhesive is sourced from one, two or more of polyvinyl pyrrolidone, hydroxypropyl methyl cellulose, hydroxypropyl cellulose, methyl cellulose, polyvinyl alcohol, and starch.

According to an embodiment of this invention, the said disintegrating agent is sourced from one, two, or more of sodium carboxymethyl cellulose, calcium carboxymethyl cellulose, crosslinked sodium carboxymethyl cellulose, crosslinked sodium carboxymethyl starch, low-substituted hydroxypropyl cellulose, crosslinked polyvinyl pyrrolidone, microcrystalline cellulose, and pre-gelatinized starch.

According to an embodiment of this invention, the said diluent is sourced from one, two or more of xylitol, mannitol, sucrose, glucose, sorbitol, maltitol, and fructose.

According to an embodiment of this invention, the said pH regulator is sourced one, two or more of organic or inorganic acids, preferable tartaric acid, citric acid, oxalic acid, succinic acid, fumaric acid, ascorbic acid, malic acid, glutamic acid, and caffeic acid.

According to an embodiment of this invention, the pH value of the suspension gel formed by the dry suspension granules in the water-based medium ranges from 2.5 to 7.0, and preferably ranges from 3.0 to 5.0.

According to an embodiment of this invention, the amount of water-bearing dispersion medium added into the dry suspension granules based on this invention is 2 to 50 times of the weight of the dry suspension granules when preparing the suspension gel.

In another aspect, this invention provides a method for preparing the dry suspension granules based on this invention, including the following steps:
(1) Mix the anionic gel and the cationic polymer as well as other optional excipients;
(2) Prepare separately the adhesive solution;
(3) Add the adhesive solution obtained in Step (2) into the mixture obtained in Step (1), to prepare the wetting mixture;
(4) Granulate the wetting mixture obtained in Step (3), to obtain the dry suspension granules based on this invention.

Preferably, the said other excipients include diluent, pH regulator, disintegrating agent, etc.

Preferably, water and/or ethanol should be used as solvent in Step (2).

In another aspect, this invention provides a method for preparing the dry suspension granules based on this invention, including the following steps:
(1) Mix the anionic gel and the cationic polymer as well as other optional excipients;
(2) Provide water and/or ethanol as the wetting agent;
(3) Add the wetting agent provided in Step (2) into the mixture obtained in Step (1), to prepare the wetting mixture;
(4) Granulate the wetting mixture obtained in Step (3), to obtain the dry suspension granules based on this invention.

Preferably, the said other excipients include diluent, pH regulator, disintegrating agent, and/or adhesive, etc.

Preferably, in the method for preparing dry suspension granules based on this invention, following Step (4) there is Step (5): Make the dry suspension granules based on this invention by drying.

Preferably, in the method for preparing the dry suspension granules based on this invention, following Step (5) there is Step (6): Granulate the dry suspension granules based on this invention in the form of pellet.

In another aspect, this invention also provides a pharmaceutical composition, in particular a dry suspension containing the dry suspension granules based on this invention.

According to an embodiment of this invention, this invention also provides a pharmaceutical composition, in particular a dry suspension, containing the dry suspension granules based on this invention and the enteric pellets, especially the enteric pellets containing Ilaprazole.

According to an embodiment of this invention, the mass ratio between the dosage of Ilaprazole in the said enteric pellets and the dosage of the dry suspension granules in the pharmaceutical composition based on this invention is 1:200-1000.

According to an embodiment of this invention, the duration in which the suspension gel obtained by adding the dispersion medium into the pharmaceutical composition based on this invention, preferably the dry suspension, reaches 75% of the maximum viscosity is less than 8 min, preferably less than 7 min.

According to an embodiment of this invention, the duration in which the suspension gel obtained by adding the dispersion medium into the pharmaceutical composition based on this invention, preferably the dry suspension, reaches 90% of the maximum viscosity is less than 12 min, preferably less than 11 min.

According to an embodiment of this invention, the duration in which the suspension gel is formed by adding the water-bearing medium into the pharmaceutical composition based on this invention, preferably the dry suspension, is less than 120 s, preferably less than 90 s, more preferably less than 70 s, and most preferably less than 50 s.

According to an embodiment of this invention, the release rate of Ilaprazole does not exceed 10% within 1 h after adding the pharmaceutical composition based on this invention, preferably the dry suspension, into the dispersion medium to form the solution with pH 1.2.

According to an embodiment of this invention, the release rate of Ilaprazole is not less than 70% within 45 min after adding the pharmaceutical composition based on this invention, preferably the dry suspension, into the dispersion medium to form the solution with pH 6.8.

In the ongoing animal experiments and studies, the Inventor discovered that the preparations containing the enteric pellets based on this invention, in particular the dry suspension, have shown beneficial effects in the treatment and/or prevention of gastrointestinal diseases; the said gastrointestinal diseases mainly include heartburn, inflammatory bowel disease, Crohn's disease, irritable bowel syndrome, ulcerative colitis, peptic ulcer, stress ulcer, bleeding peptic ulcer, duodenal ulcer and recurrent duodenal ulcer, gastric ulcer associated with NSAID, adult active benign gastric ulcer, infective enteritis, colitis, hyperacidity, dyspepsia, gastroparesis, Zollinger-Ellison syndrome, gastroesophageal reflux disease (GERD), diseases related to helicobacter pylori or eradication of helicobacter pylori, erosive esophagitis and short bowel syndrome at all levels, or any combination of the above diseases.

Therefore, according to one aspect of this invention, this invention provides a method for treatment and/or prevention of gastrointestinal diseases. The method includes the steps for administering effective dose of the dry suspension based on this invention for treatment and/or prevention to the patients who need such treatment and/or prevention. Among others, the gastrointestinal diseases that can be treated and/or prevented by this method include but are not limited to heartburn, inflammatory bowel disease, Crohn's disease, irritable bowel syndrome, ulcerative colitis, peptic ulcer, stress ulcer, bleeding peptic ulcer, duodenal ulcer and recurrent duodenal ulcer, gastric ulcer associated with NSAID, adult active benign gastric ulcer, infective enteritis, colitis, hyperacidity, dyspepsia, gastroparesis, Zollinger-Ellison syndrome, gastroesophageal reflux disease (GERD), diseases related to helicobacter pylori or eradication of helicobacter pylori, erosive esophagitis and short bowel syndrome at all levels, or any combination of the above diseases.

While according to another aspect of this invention, this invention provides the use of the dry suspension based on this invention in the preparation of drugs for treatment and/or prevention of gastrointestinal diseases; the said gastrointestinal diseases include but are not limited to heartburn, inflammatory bowel disease, Crohn's disease, irritable bowel syndrome, ulcerative colitis, peptic ulcer, stress ulcer, bleeding peptic ulcer, duodenal ulcer and recurrent duodenal ulcer, gastric ulcer associated with NSAID, adult active benign gastric ulcer, infective enteritis, colitis, hyperacidity, dyspepsia, gastroparesis, Zollinger-Ellison syndrome, gastroesophageal reflux disease (GERD), diseases related to helicobacter pylori or eradication of helicobacter pylori, erosive esophagitis and short bowel syndrome at all levels, or any combination of the above diseases.

Further, when the dry suspension based on this invention is used for preparation of the drugs for treatment and/or prevention of the gastrointestinal diseases, the said gastrointestinal diseases include but are not limited to duodenal ulcer and recurrent duodenal ulcer, gastric ulcer, gastroesophageal reflux disease (GERD), diseases related to helicobacter pylori; or, the said drugs may be used to eradicate helicobacter pylori, prevent peptic ulcer diseases caused by non-steroidal anti-inflammatory drugs, and prevent gastrointestinal bleeding and related ulcers caused by anti-platelet aggregation drugs (including but not limited to clopidogrel, prasugrel, and ticagrelor).

### DESCRIPTION OF THE DRAWINGS

FIG. 1 shows the duration in which the dry suspension granules I-III based on this invention, the dry suspension granules IV based on prior art, and the dry suspension granules V in contrast, reach the maximum percentage viscosity after water is added as the dispersion medium.

FIG. 2 shows the pellet suspension-sedimentation time of the dry suspension within the volume of 0-5 mL measured in a 15 mL volumetric cylinder, which is prepared by adding different pellets into the dry suspension granules I-III based on this invention and the dry suspension granules IV based on prior art.

FIG. 3 shows the pellet suspension-sedimentation time of the dry suspension within the volume of 5-10 mL measured in a 15 mL volumetric cylinder, which is prepared by adding different pellets into the dry suspension granules I-III based on this invention and the dry suspension granules IV based on prior art.

FIG. 4 shows the pellet suspension-sedimentation time of the dry suspension within the volume of 10-15 mL measured in a 15 mL volumetric cylinder, which is prepared by adding different pellets into the dry suspension granules I-III based on this invention and the dry suspension granules IV based on prior art.

### BENEFICIAL EFFECTS OF THE INVENTION

Compared with prior art, the dry suspension granules based on this invention and the dry suspension containing such dry suspension granules can disperse rapidly in the water-bearing medium to form a stable suspension gel, and obtain a uniform and stable dispersion system containing suspended pellets with the coexistence of pellets; the resulting solution/gel can reach the stable viscosity in a shorter period of time than the dry suspension granules and the dry suspension based on prior art, and maintain the pellets in a stable state of suspension for a longer period of time.

For the acid-unstable proton pump inhibitors, especially Ilaprazole, it is possible to increase their dissolution rate with the suspension solution obtained from the dry suspension based on this invention.

### SPECIFIC EMBODIMENTS

In the preferable embodiment based on this invention, the dry suspension based on this invention contains the enteric pellets, preferable Ilaprazole enteric pellets, and dry suspension granules based on this invention. The dry suspension based on this invention may contain, but without limitation to, the following components in parts by weight:
Enteric pellets 1-30;
Gel 0.1-15;
Adhesive 0.1-20;
Disintegrating agent 0.1-15;
Diluent 1-90;
pH regulator 0.02-6;
Cationic polymer 0.1-15.

In terms of the enteric pellets, the pellets based on prior art may be used or the enteric pellets in the following embodiments may be used preferably according to the various aspects of this invention.

According to this invention, in the first embodiment of the enteric pellets, such enteric pellets include the pellet core, the first isolating layer, the second isolating layer, and the enteric layer from inside to outside; among them, the pellet core contains Ilaprazole and/or salt pharmaceutically acceptable to Ilaprazole and the first excipient; the said enteric pellets are characterized by the fact that there is water insoluble alkaline compound in the first isolating layer; the weight ratio between the first excipient and Ilaprazole and/or salt pharmaceutically acceptable to Ilaprazole is 0.2-5:1.

According to this invention, in the second embodiment of the enteric pellets, such enteric pellets include the pellet core, the first isolating layer, the second isolating layer, and the enteric layer from inside to outside; among them, the pellet core contains Ilaprazole and/or salt pharmaceutically acceptable to Ilaprazole and the first excipient; the said enteric pellets are characterized by the fact that there is water insoluble alkaline compound in the first isolating layer and the first excipient is water insoluble alkaline compound; the water insoluble alkaline compound in the first isolating layer may be the same as or different from the water insoluble alkaline compound used as the first excipient.

According to this invention, in the third embodiment of the enteric pellets, such enteric pellets include the pellet core, the first isolating layer, the second isolating layer, and the enteric layer from inside to outside; among them, the pellet core contains Ilaprazole and/or salt pharmaceutically acceptable to Ilaprazole and the first excipient; the said enteric pellets are characterized by the fact that the particle size (D90) of Ilaprazole and/or salt pharmaceutically acceptable to Ilaprazole is less than or equal to 100 μm, and there is no alkaline substance in the second isolating layer.

Preferably, there is a protective layer on the outside of the enteric layer of the enteric pellets based on this invention.

Preferably, there is no other layer between the pellet core and the first isolating layer of the enteric pellets based on this invention.

Preferably, there is no other layer between the second isolating layer and the enteric layer of the enteric pellets based on this invention.

Preferably, there is no other layer between the first isolating and the second isolating layer of the enteric pellets based on this invention.

Preferably, the first excipient in the enteric pellets based on this invention is an alkaline compound, preferably a water insoluble alkaline compound, and more preferably magnesium hydroxide, aluminum hydroxide, magnesium oxide, magnesium carbonate, calcium carbonate, and calcium hydroxide.

Preferably, the salt pharmaceutically acceptable to Ilaprazole may be, for example, Ilaprazole sodium, Ilaprazole magnesium, Ilaprazole zinc, Ilaprazole potassium, Ilaprazole lithium, or Ilaprazole calcium. For this purpose, the technical personnel in the field may select the appropriate salt according to their needs.

Preferably, the pellet core of the enteric pellets based on this invention also contains surfactant. Preferably, the said surfactant is Tween 80 or sodium dodecyl sulfate.

Preferably, the weight ratio between the water insoluble alkaline compound in the first isolating layer and Ilaprazole and/or salt pharmaceutically acceptable to Ilaprazole is 0.2-5:1, preferably 0.25-4:1, more preferably 0.3-3:1, particularly preferably 0.5-2:1, and most preferably 0.8-1.2:1, e.g. 1:1.

Preferably, in the second isolating layer, there is the water insoluble inert substance that can prevent the adhesion of pellets, and the dosage ratio between such water insoluble inert substance and the adhesive by weight ranges from 1-8:1.5-10, or 1-10:1-20, or 4-26:7-44.

Preferably, the particle size (D90) of Ilaprazole and/or salt pharmaceutically acceptable to Ilaprazole may be selected from the range between any two of the following endpoints: 0 μm (excluding the value of this point when constituting the range) 10 μm, 20 μm, 30 μm, 40 μm, 50 μm, 60 μm, 70 μm, 80 μm, 90 μm, and 100 μm.

### Pellet Core of Enteric Pellets

The pellet core (or known as drug-containing pellet) of the enteric pellets based on this invention may be a fully active pellet core or a blank pellet core coated with a drug loading layer. The term “fully active pellet core” referred to herein means the pellet core containing Ilaprazole and/or salt pharmaceutically acceptable to Ilaprazole and the first excipient, as well as one or more other pharmaceutical excipients; among others, Ilaprazole and/or salt pharmaceutically acceptable to Ilaprazole serving as the active ingredients are dispersed in other ingredients (the said first excipient and one or more other pharmaceutical excipients, etc.), but do not form additional layers independently or together with any other ingredients; in the blank pellet core coated with a drug loading layer, the drug loading layer contains Ilaprazole and/or salt pharmaceutically acceptable to Ilaprazole and the first excipient as well as optional excipient.

If Ilaprazole and/or salt pharmaceutically acceptable to Ilaprazole in the pellet core cannot achieve sufficient storage stability only with the action of the water insoluble alkaline compound contained in the first isolating layer, its storage stability may be improved by adding more quantity of the first excipient into the pellet core.

The first excipient in the pellet core may be a conventional excipient based on prior art and used to improve the stability of acid-unstable compounds. Preferably, the first excipient is the alkaline compounds, including water insoluble alkaline compounds and water soluble alkaline compounds. Preferably based on this invention, a water insoluble alkaline compound is used as the first excipient in the pellet core; more preferably, the water insoluble alkaline compound contained in the pellet core is the same as the water insoluble alkaline compound contained in the first isolating layer, so that it is possible to further enhance the buffering effect of the isolating layer on pH value. In the embodiments based on this invention, the water insoluble alkaline compounds may be selected from, but are not limited to, one or more of magnesium hydroxide, aluminum hydroxide, magnesium oxide, magnesium carbonate, calcium carbonate, and calcium hydroxide. Preferably, the weight ratio between the alkaline compound and Ilaprazole and/or salt pharmaceutically acceptable to Ilaprazole is 0.2-5:1, preferably 0.25-4:1, more preferably 0.3-3:1, particularly preferably 0.5-2:1, and most preferably 0.8-1.2:1, e.g. 1:1.

According to this invention, surfactant may also be contained in the pellet core. It is proved by the embodiments based on this invention that the surfactant can increase the dissolution rate of Ilaprazole and/or salt pharmaceutically acceptable to Ilaprazole in the enteric pellets and their preparations, so as to improve their bioavailability effectively. In the embodiments based on this invention, the surfactant contained in the pellet core may be selected from non-ionic surfactant, anionic surfactant, and amphoteric surfactant. Preferably, the non-ionic surfactant may be selected from polyethylene glycol, polyol (e.g. Tween 80), etc. The anionic surfactant may be selected from higher fatty acid salts, sulfate ester salts, and sulfonates such as sodium dodecyl sulfate; the amphoteric surfactant may be selected from phosphorus esters.

According to this invention, the particle size of Ilaprazole and/or salt pharmaceutically acceptable to Ilaprazole may affect the dissolution rate and/or drug loading capacity of the enteric pellets. In the preferable embodiment based on this invention, the particle size (D90) of Ilaprazole and/or salt pharmaceutically acceptable to Ilaprazole may be less than or equal to 100 μm; in this case, enteric pellets have good dissolution rate, and the bioavailability of enteric pellet preparations made of enteric pellets may be improved. More preferably, the particle size (D90) of Ilaprazole and/or salt pharmaceutically acceptable to Ilaprazole may be selected from the range between any two of the following endpoints: 10 μm, 20 μm, 30 μm, 40 μm, 50 μm, 60 μm, 70 μm, 80 μm, 90 μm, and 100 μm; in particular when D90 is less than or equal to 50 μm, it is possible to achieve the improved drug loading capacity.

According to an embodiment of this invention, the abovementioned blank pellet core is the blank pellet core conventionally used in prior art. In the embodiments based on this invention, the blank pellet core may be selected from, but is not limited to, the microcrystalline cellulose pellet core, the sucrose pellet core, or the mannitol pellet core with the particle size of 50-500 μm, preferably 100-400 μm, more preferably 250-350 μm, and most preferably about 300 μm.

According to an embodiment of this invention, the abovementioned drug loading layer may also contain adhesive. The adhesive may be selected from, but is not limited to, one or more of hydroxypropyl cellulose, hydroxypropyl methyl cellulose, hydroxymethyl cellulose, hydroxyethyl cellulose, carboxymethyl cellulose, methyl cellulose, polyvinyl pyrrolidone, polyvinyl alcohol, polyethylene glycol, syrup, and starch. In the embodiments based on this invention, the adhesive may be one or more of hydroxypropyl cellulose-SSL (e.g. commercially available series from Nisso), hydroxypropyl methyl cellulose E5, polyvinyl pyrrolidone K30, polyvinyl alcohol, methyl cellulose, and polyethylene glycol.

### Isolating Layers of Enteric Pellets

The basic function of the isolating layers of enteric pellets is to isolate the pellet core in an alkaline environment from the enteric layer containing free carboxyl group, in order to prevent degradation or discoloration of Ilaprazole and/or salt pharmaceutically acceptable to Ilaprazole during the process of coating or storage. In the research on Ilaprazole enteric pellets and their preparations, the Inventor discovered that the water insoluble inert substance (e.g. talcum powder, silicon dioxide, titanium dioxide, magnesium stearate) and/or the water soluble alkaline compounds commonly used in conventional preparation method (e.g. when preparing the isolating layer closely adjacent to the pellet core) based on prior art to prevent adhesion of pellets, as well as the alkaline compounds commonly used in preparation of the isolating layer closely adjacent to the enteric layer, can reduce the stability and/or acid resistance of the enteric pellets and their preparations; in particular, for the acid-unstable compounds with relatively low stability such as Ilaprazole, the enteric pellets and their preparations cannot meet the requirements for stability and acid resistance simultaneously as a result. The term “closely adjacent” referred to herein means that there is no additional layer between the pellet core of enteric pellets and its coating or cladding, or between any two layers.

As one of the causes for this technical problem, it is proved by test and believed by the Inventor that (but without limitation to) the compatibility between the water insoluble inert substance contained in the isolating layer (corresponding to the first isolating layer based on this invention) closely adjacent to the pellet core to prevent adhesion of pellets and the acid-unstable compound contained in the pellet core differs due to the different stability of the acid-unstable compound; that is to say, in the enteric pellets and their preparations prepared according to prior art, if the stability of the acid-unstable compound (e.g. Ilaprazole) contained in the pellet core is low, the compatibility between the water insoluble inert substance (e.g. talcum powder) contained in the isolating layer closely adjacent to the pellet core to prevent adhesion of pellets and the acid-unstable compound will also be reduced.

Therefore, even under the protection of alkaline compound contained as stabilizer in the pellet core and/or the isolating layer, there is still a significant increase in the relevant substances (i.e. impurities) in the accelerated test results, thus reducing the stability of enteric pellet preparations. This will limit, to some extent, the range of the acid-unstable compounds applicable to the enteric pellets prepared according to prior art and their preparations, that is to say, the prescription or the components of enteric pellets prepared according to prior art and their preparations cannot be applied properly to Ilaprazole and/or salt pharmaceutically acceptable to Ilaprazole with relatively low stability. In addition, if the isolating layer (corresponding to the first isolating layer based on this invention) closely adjacent to the pellet core contains water soluble alkaline compound, the isolating layer of the enteric pellets will absorb free water and cause dissolution of the water soluble alkaline compound in the condition of high temperature and high humidity for a long time, so that the alkalinity of the isolating layer closely adjacent to the enteric layer will increase and the isolating layer becomes alkaline; if water infiltrates into the enteric layer with the acidic medium, the enteric layer will dissolve in advance, resulting in the decrease in acid resistance of enteric pellets and their preparations. The principle that the enteric layer will dissolve in advance because the isolating layer closely adjacent to the enteric layer becomes alkaline is also applicable to the situation that the isolating layer (corresponding to the second isolating layer based on this invention) closely adjacent to the enteric layer contains alkaline compound.

Therefore, according to this invention, the enteric pellets include at least two isolating layers containing inert substance, i.e. the first isolating layer closely adjacent to the pellet core, and the second isolating layer which is far away from the pellet core compared with the first isolating layer or is closely adjacent to the enteric layer. Among them, the first isolating layer contains water insoluble alkaline compound, but does not contain water soluble alkaline compound and water insoluble inert substance that can prevent adhesion of pellets, and the second isolating layer does not contain alkaline compound. If the enteric pellets include three or more isolating layers, the other isolating layer between the first and the second isolating layers may be an isolating layer conforming to the definition of the first or the second isolating layer based on this invention, or an isolating layer commonly used in prior art.

According to this invention, the water insoluble alkaline compound may be the water insoluble alkaline compound commonly used in prior art to improve the stability of acid-unstable compound. In the embodiments based on this invention, the water insoluble alkaline compound may be selected from, but is not limited to, one or more of magnesium hydroxide, aluminum hydroxide, magnesium oxide, magnesium carbonate, calcium carbonate, and calcium hydroxide.

According to this invention, the water insoluble inert substance that can prevent adhesion of pellets may be selected from one or more of lubricants, flow aids and anti-adherents (i.e. anti-adhesion agents, the same below) conventionally used in pharmacy. In the embodiments based on this invention, the water insoluble inert substance that can prevent adhesion of pellets may be selected from, but is not limited to, silicon dioxide, calcium silicate, colloidal silicon dioxide, aluminum silicate, aluminum calcium silicate, magnesium silicate, sodium stearate, zinc stearate, magnesium stearate, talcum powder, and titanium dioxide. In an embodiment of Ilaprazole enteric pellets based on this invention, the first isolating layer of the enteric pellets does not contain one or more of the following water insoluble inert substances that can prevent adhesion of pellets, such as talcum powder, silicon dioxide, titanium dioxide, and magnesium stearate.

In the preferable embodiment based on this invention, the first isolating layer is mainly composed of water insoluble alkaline compound and adhesive, and the second isolating layer is mainly composed of water insoluble inert substance that can prevent adhesion of pellets and adhesive. According to this invention, by adjusting the dosage of water insoluble alkaline compound and adhesive contained in the first isolating layer or the dosage of water insoluble inert substance that can prevent adhesion of pellets and adhesive contained in the second isolating layer, it is possible to affect the dissolution rate of the enteric pellet preparations, thus affecting their bioavailability. For example, in the preferable embodiment of Ilaprazole enteric pellet tablets based on this invention, the ratio of the components are shown as follows: if the dosage of Ilaprazole is 5-15 parts by weight, the first isolating layer contains 5-36 parts of adhesive by weight and 5-36 parts of water insoluble alkaline compound by weight; the second isolating layer contains 4-26 parts of adhesive by weight and 7-44 parts of water insoluble inert substance by weight that can prevent adhesion of pellets.

The abovementioned adhesives are commonly used in isolating layers based on prior art. According to this invention, the adhesives may be selected from qualified medicinal water soluble inert compound or the polymer used as coating film, such as one or more of hydroxypropyl cellulose, hydroxypropyl methyl cellulose, hydroxymethyl cellulose, hydroxyethyl cellulose, carboxymethyl cellulose, methyl cellulose, polyvinyl pyrrolidone, polyvinyl alcohol, polyethylene glycol, syrup, and starch.

### Enteric Layer of Enteric Pellets

The enteric layer commonly used in enteric pellet preparations based on prior art is also applicable to this invention; for example, a detailed introduction to enteric layer is recorded in CN87103285A (Chinese homdogy of U.S. Pat. No. 4,786,505); the Inventor used it as a reference and included the contents related to the enteric layer in the said literature and the relevant contents of the literature cited therein into this application.

According to this invention, the enteric layer may contain one or more substances selected from the following group: acrylic resins, cellulose such as carboxymethyl ethyl cellulose, enteric coating materials such as Opadry, and one or more additives selected optionally from plasticizer, anti-adherent and lubricant. In the embodiments based on this invention, the enteric layer may contain acrylic resin enteric coating material, plasticizer (e.g. polyethylene glycol, glyceryl triacetate, triethyl citrate, phthalate), and anti-adherent (e.g. talcum powder, glyceryl monostearate). Among them, acrylic resin enteric coating material is selected from one or more of cellulose acetate phthalate, hydroxypropyl methyl cellulose phthalate, solution or dispersion liquid L30D55 of methacrylic acid copolymer, hydroxypropyl methyl cellulose acetate succinate, polyvinyl acetate phthalate, and shellac. In the preferable embodiment of Ilaprazole enteric pellets based on this invention, the weight ratio between the enteric coating material contained in the enteric layer and Ilaprazole and/or salt pharmaceutically acceptable to Ilaprazole is 2-20:1. In the preferable embodiment of Ilaprazole enteric pellets based on this invention, the weight ratio between the plasticizer and Ilaprazole and/or salt pharmaceutically acceptable to Ilaprazole is 0.6-6:1, preferably 0.8-4:1, and more preferably 1-2:1.

### Protective Layer of Enteric Pellets

Based on this invention, there may be a protective layer on the outside of the enteric layer of the enteric pellets, and preferably the protective layer is closely adjacent to the enteric layer. The said protective layer can prevent the adhesion that may occur between various semi-finished products/products during the placement before they are made into preparations, or during the making of their preparations, or during the placement after they are made into preparations. In addition, the protective layer can increase the dissolution rate of enteric pellets effectively, and thus improve the bioavailability of enteric pellet preparations made with enteric pellets.

In the embodiments based on this invention, the protective layer may contain adhesive and anti-adherent. The adhesive may be selected from one or more of hydroxypropyl methyl cellulose, hydroxypropyl cellulose, polyvinyl pyrrolidone, polyvinyl alcohol, methyl cellulose, and polyethylene glycol. The anti-adherent may be selected from one or more of talcum powder, magnesium stearate, titanium dioxide, and silicon dioxide.

Based on this invention, it is possible to improve the acid resistance of the enteric pellets by increasing the dosage of the anti-adherent in the protective layer. In the preferable embodiment of Ilaprazole enteric pellets based on this invention, if Ilaprazole and/or salt pharmaceutically acceptable to Ilaprazole is 5-15 parts by weight, the dosage of anti-adherent is 0.5-5 parts by weight.

According to the fourth aspect of this invention, this invention provides the preparation method for enteric pellets. The said preparation method at least includes the following steps: 1) Prepare the pellet core containing Ilaprazole and/or salt pharmaceutically acceptable to Ilaprazole and the first excipient; 2) Coat the first isolating layer, and then the second isolating layer; and 3) Coat the enteric layer.

Preferably, Step 2) includes the following: Prepare the first suspension containing water insoluble alkaline compound but not containing water soluble alkaline compound and the water insoluble inert substance that can prevent adhesion of pellets, and coat the first suspension on the pellet core obtained in Step 1); prepare the second suspension not containing alkaline compound, and coat the second suspension as the second isolating layer, preferably coat the second suspension as the second isolating layer closely adjacent to the enteric layer.

Preferably, in Step 1), the said first excipient interacts with the water insoluble alkaline compound contained in the said first isolating layer, so as to achieve the storage stability of Ilaprazole and/or salt pharmaceutically acceptable to Ilaprazole.

Preferably, the preparation method for enteric pellets also includes Step 4): Coat the protective layer.

In the embodiments based on this invention, the preparation method for enteric pellets may include one or more of the following steps:
1) Coat the blank pellet core with a drug loading layer by the fluidized bed method and others, and prepare the pellet core; the drug loading layer includes Ilaprazole and/or salt pharmaceutically acceptable to Ilaprazole, the alkaline compound as the first excipient, and the adhesive;
2) By the fluidized bed method and others, coat at least the first suspension and the second suspension respectively from inside to outside on the pellet core obtained in Step 1); among them, the first suspension contains water insoluble alkaline compound, but does not contain water soluble alkaline compound and the water insoluble inert substance that can prevent adhesion of pellets, forming the first isolating layer; the second suspension does not contain alkaline compound, forming the second isolating layer; the isolating pellets are prepared by this method.
3) Make the enteric layer suspension with the enteric coating material and one or more of plasticizer, anti-adherent, lubricant and emulsifier; by the fluidized bed method and others, coat the enteric layer suspension on the isolating pellets obtained in Step 2), so as to prepare the enteric pellets.

Preferably, the abovementioned preparation method for enteric pellets may also include Step 4): Add one or more adhesives selected from hydroxypropyl cellulose, hydroxypropyl methyl cellulose, methyl cellulose, polyvinyl pyrrolidone, polyvinyl alcohol, carboxymethyl cellulose, and polyethylene glycol into purified water, and prepare the coating solution of protective layer, and coat the coating solution of protective layer on the enteric pellets obtained in Step 3) by the fluidized bed method and others, so as to prepare the enteric pellets with protective layer.

Taking the dry suspension as an example, the preparation methods and the property test experiments for the enteric pellets based on this invention and the dry suspension containing such enteric pellets are described below.

### EMBODIMENTS

### Embodiment 1: Preparation of Pellets

First, in this embodiment, the enteric pellets A, D, and E based on this invention are prepared by taking Ilaprazole and/or salt pharmaceutically acceptable to Ilaprazole as an example.

### (I) Preparation Prescription for the Component Layers of Pellets

1.1 Preparation and Drug Application Rate of Drug-Containing Pellet (i.e. Pellet Core W)

(1) 150 g of hydroxypropyl cellulose-SSL and 4 g of polysorbate 80 are weighed and dissolved in 3000 g of purified water to obtain hydroxypropyl cellulose adhesive solution, and 100 g of magnesium hydroxide is added into the adhesive solution and dispersed with high shear at 10000 rpm for 5 min, and then 100 g of Ilaprazole with particle size D90 of 46.8 μm is dispersed into the adhesive solution containing magnesium hydroxide and dispersed uniformly with high shear at 10000 rpm; Ilaprazole suspension is sprayed onto 100 g of sucrose pellet cores in GLATT GPCG-1 fluidized bed to make the drug-containing pellet W1.

The process parameters of the fluidized bed are shown as follows:

| Parameter | Preheating | Drug Application | Drying |
|---|---|---|---|
| Air intake rate (m³/min) | 45 | 50-80 | 60 |
| Air intake temperature (° C.) | 40-50 | 40-50 | 40-50 |
| Temperature of material (° C.) | 38-40 | 35-40 | 35-40 |
| Atomization pressure (bar) | 0.1 | 0.1-0.2 | 0.1 |

| | |
|---|---|
| Spraying rate (g/min) | 0.5-4 |

Where, the drug application rate of drug-containing pellet W1=(Actual content of drug-containing pellet)/(Theoretical content of drug-containing pellet)×100%=95.6%

(2) 180 g of polyvinyl pyrrolidone K30 and 6 g of polysorbide 80 are weighed and dissolved in 3000 g of purified water, and 50 g of magnesium oxide is added and dispersed uniformly with high shear at 10000 rpm to obtain the adhesive containing magnesium oxide; then 150 g of Ilaprazole is dispersed into the adhesive containing magnesium oxide and dispersed uniformly with high shear at 10000 rpm; Ilaprazole suspension is sprayed onto 100 g of mannitol pellet cores in GLATT GPCG-1 fluidized bed to make the drug-containing pellet W4.

The process parameters of the fluidized bed are shown as follows:

| Parameter | Preheating | Drug Application | Drying |
|---|---|---|---|
| Air intake rate (m³/min) | 50 | 50-70 | 60 |
| Air intake temperature (° C.) | 40-50 | 45-55 | 45-55 |
| Temperature of material (° C.) | 38-40 | 35-40 | 35-40 |
| Atomization pressure (bar) | 0.1 | 0.1-0.2 | 0.1 |

| | |
|---|---|
| Spraying rate (g/min) | 0.5-4 |

Where, the drug application rate of drug-containing pellet W4=(Actual content of drug-containing pellet)/(Theoretical content of drug-containing pellet)×100%=93.3%

(3) 80 g of hydroxypropyl methyl cellulose E5 and 6 g of polysorbide 80 are weighed and dissolved in 1600 g of purified water, and 50 g of magnesium hydroxide is added and dispersed uniformly with high shear at 10000 rpm to obtain the adhesive containing magnesium hydroxide; then 150 g of Ilaprazole is dispersed into the adhesive containing magnesium hydroxide and dispersed uniformly with high shear at 10000 rpm; Ilaprazole suspension is sprayed onto 150 g of sucrose pellet cores in GLATT GPCG-1 fluidized bed to make the drug-containing pellet W5.

The process parameters of the fluidized bed are shown as follows:

| Parameter | Preheating | Drug Application | Drying |
|---|---|---|---|
| Air intake rate (m³/min) | 50 | 50-70 | 60 |
| Air intake temperature (° C.) | 40-50 | 45-55 | 45-55 |
| Temperature of material (° C.) | 38-40 | 35-40 | 35-40 |
| Atomization pressure (bar) | 0.1 | 0.1-0.2 | 0.1 |

| | |
|---|---|
| Spraying rate (g/min) | 0.5-4 |

Where, the drug application rate of drug-containing pellet W5=(Actual content of drug-containing pellet)/(Theoretical content of drug-containing pellet)×100%=94.1%

### 1.2 Preparation of the Isolating Layer (G)

23 g of hydroxypropyl cellulose-SSL is weighed and dissolved in 460 g of purified water, and then 23 g of magnesium carbonate is added and dispersed uniformly with high shear at 10000 rpm to prepare the prescription for the first isolating layer. 17.2 g of hydroxypropyl cellulose-SSL is weighed and dissolved in 344 g of purified water, and 28.8 g of talcum powder is added and dispersed uniformly with high shear at 10000 rpm to prepare the prescription for the second isolating layer. The two layers of isolating suspension are sprayed respectively onto 90 g of corresponding pellet cores in GLATT GPCG-1 fluidized bed.

The process parameters of the fluidized bed are shown as follows:

| Parameter | Preheating | Isolating Coating | Drying |
|---|---|---|---|
| Air intake rate (m³/min) | 50 | 50-60 | 60 |
| Air intake temperature (° C.) | 40-50 | 45-55 | 45-55 |
| Temperature of material (° C.) | 38-40 | 35-40 | 35-40 |
| Atomization pressure (bar) | 0.1 | 0.1-0.2 | 0.1 |

| | |
|---|---|
| Spraying rate (g/min) | 0.5-3.5 |

### 1.3 Preparation of the Enteric Layer (C)

### Enteric Layer Prescription C (Unit: g)

| | |
|---|---|
| Ilaprazole isolating pellet | 92 |
| Eudragit L30D-55 | 223.8 |
| Talcum powder | 3.4 |
| Triethyl citrate | 20.1 |
| Purified water | 447.6 |

### Process of Preparation:

20.1 g of triethyl citrate is weighed and dissolved in 447.6 g of purified water, and 3.4 g of talcum powder is added and dispersed uniformly with high shear at 10000 rpm; and then the solution is stirred with 223.8 g of Eudragit L30D-55 for 45 min for standby, and the enteric coating solution is sprayed onto the isolating pellets in GLATT GPCG-1 fluidized bed.

The specific process parameters are shown as follows:

| Parameter | Preheating | Enteric Coating | Drying |
|---|---|---|---|
| Air intake rate (m³/min) | 50 | 50-80 | 60 |
| Air intake temperature (° C.) | 35-30 | 33-42 | 35-45 |
| Temperature of material (° C.) | 25-30 | 25-30 | 30-35 |
| Atomization pressure (bar) | 0.1 | 0.15-0.25 | 0.1 |

| | |
|---|---|
| Spraying rate (g/min) | 0.5-3 |

### 1.4 Preparation of the Protective Layer (B)

### 1) Protective Layer Prescription B1 (Unit: g)

| | |
|---|---|
| Ilaprazole enteric pellets | 100 |
| Hydroxypropyl methyl cellulose E5 | 2.5 |
| Magnesium stearate | 1.2 |
| Purified water | 50 |

### Process of Preparation:

2.5 g of hydroxypropyl methyl cellulose E5 in prescription dosage is weighed and dissolved in 50 g of purified water, and then 1.2 g of magnesium stearate is added and dispersed uniformly with high shear at 5000 rpm to make the coating solution of protective layer; the coating solution of protective layer is sprayed onto the enteric pellets in GLATT GPCG-1 fluidized bed.

The specific process parameters are shown as follows:

| Parameter | Preheating | Isolating Coating | Drying |
|---|---|---|---|
| Air intake rate (m³/min) | 50 | 50-60 | 60 |
| Air intake temperature (° C.) | 40-50 | 45-55 | 45-55 |
| Temperature of material (° C.) | 38-40 | 35-40 | 35-40 |
| Atomization pressure (bar) | 0.1 | 0.1-0.2 | 0.1 |

| | |
|---|---|
| Spraying rate (g/min) | 0.5-3.5 |

### 2) Protective Layer Prescription B4 (Unit: g)

| | |
|---|---|
| Ilaprazole enteric pellets | 100 |
| Hydroxypropyl cellulose-SSL | 2.5 |
| Talcum powder | 1.2 |
| Purified water | 50 |

### Process of Preparation:

2.5 g of hydroxypropyl cellulose-SSL in prescription dosage is weighed and dissolved in 50 g of purified water, and then 1.2 g of talcum powder is added and dispersed uniformly with high shear at 5000 rpm to make the coating solution of protective layer. The coating solution of protective layer is sprayed onto the enteric pellets in GLATT GPCG-1 fluidized bed.

Please see the abovementioned protective layer prescription B1 for the specific process parameters.

### 3) Protective Layer Prescription B5 (Unit: g)

| | |
|---|---|
| Ilaprazole enteric pellets | 100 |
| Hydroxypropyl cellulose-SSL | 2.5 |
| Titanium dioxide | 1.2 |
| Purified water | 50 |

Process of preparation:

2.5 g of hydroxypropyl cellulose-SSL in prescription dosage is weighed and dissolved in 50 g of purified water, and then 1.2 g of titanium dioxide is added and dispersed uniformly with high shear at 5000 rpm to make the coating solution of protective layer. The coating solution of protective layer is sprayed onto the enteric pellets in GLATT GPCG-1 fluidized bed.

Please see the abovementioned protective layer prescription B1 for the specific process parameters.

### (II) Preparation of Enteric Pellets

According to the prescriptions for the component layers of enteric pellets as set forth in Section (I) of this embodiment, the enteric pellets A, D and E based on this invention are prepared as shown in Table 1 below.

TABLE 1
Prescription Components of Enteric
Pellets Based on This Invention

| | Pellet A | Pellet D | Pellet E |
|---|---|---|---|
| drug-containing pellet | W1 | W4 | W5 |
| Isolating layer | G2 | G2 | G2 |
| Enteric layer | C1 | C1 | C1 |
| Protective layer | B1 | B4 | B5 |
| Drug loading capacity of | 95.6 | 93.3 | 94.1 |
| drug-containing pellet (%) | | | |

Where, Wₓ drug application rate of drug-containing pellet=(Actual content of drug-containing pellet)/(Theoretical content of drug-containing pellet)×100%

### Embodiment 2: Preparation of Pellet Dry Suspension

In this embodiment, first the dry suspension granules are prepared, and then different pellets are added to prepare the dry suspension containing pellets.

### 2.1 Preparation of Dry Suspension A-I

### 1) Preparation of Dry Suspension Granules

| Prescription | Dosage |
|---|---|
| Carrageenan | 25 g |
| Hydroxypropyl cellulose LF | 10 g |
| Crosslinked povidone | 25 g |
| Mannitol | 700 g |
| Glucose | 250 g |
| Malic acid | 4 g |
| Chitosan | 25 g |
| Purified water | 190 g |

Preparation method: 10 g of hydroxypropyl cellulose LF is weighed and dissolved in 190 g of purified water, and the prescription dosage of carrageenan, crosslinked povidone, chitosan, mannitol, glucose, and malic acid are weighed and delivered into HLSH2-6 wet mixing granulator for stirring and mixing at 400 rpm for 4 min, and then hydroxypropyl cellulose LF is dissolved and added into the mixer for further stirring for 3 min. The shearing is conducted at 800 rpm and continued for 1 min before discharging, so as to prepare the wet granules; the wet granules are dried and granulated with 1.2 mm mesh, so as to obtain the dry suspension granules I.

### 2) Preparation of Dry Suspension

94.5 mg of Ilaprazole enteric pellet A and 1.5 g of the abovementioned dry suspension granules are mixed to obtain the dry suspension A-I.

### 2.2 Preparation of Dry Suspension D-II

### 1) Preparation of Dry Suspension Granules

| Prescription | Dosage |
|---|---|
| Arabic gum | 20 g |
| Sodium alginate | 10 g |
| Hydroxypropyl methyl cellulose E5 | 15 g |
| Crosslinked sodium carboxymethyl cellulose | 30 g |
| Mannitol | 600 g |
| Sucrose | 400 g |
| Citric acid | 3.5 g |
| Chitosan | 10 g |
| Purified water | 210 g |

Preparation method: The prescription dosage of Arabic gum, sodium alginate, hydroxypropyl methyl cellulose E5, crosslinked sodium carboxymethyl cellulose, mannitol, sucrose, citric acid, and chitosan are weighed and delivered into HLSH2-6 wet mixing granulator, and then stirred and mixed at 400 rpm for 5 min, and the purified water is added slowly while stirring, and the solution is further stirred for 5 min. The shearing is conducted at 700 rpm and continued for 1 min to make the wet granules, which are discharged, dried, and granulated with 1.0 mm mesh, so as to obtain the dry suspension granules II.

### 2) Preparation of Dry Suspension

67.4 mg of Ilaprazole enteric pellet D and 2.5 g of the abovementioned dry suspension granules are mixed and bottled to obtain Ilaprazole enteric dry suspension D-II in the specification of 5 mg.

### 2.3 Preparation of Dry Suspension E-III

### 1) Preparation of Dry Suspension Granules III

| Prescription | Dosage |
|---|---|
| Pectin | 30 g |
| Sodium alginate | 5 g |
| Polyvinyl pyrrolidone K30 | 10 g |
| Crosslinked povidone | 35 g |
| Maltose | 200 g |
| Xylitol | 900 g |
| Ascorbic acid | 9 g |
| Chitosan | 35 g |
| Purified water | 190 g |

Preparation method: 10 g of polyvinyl pyrrolidone K30 is weighed and dissolved in 190 g of purified water, and the prescription dosage of pectin, sodium alginate, crosslinked povidone, maltose, xylitol, ascorbic acid, and chitosan are weighed and delivered into HLSH2-6 wet mixing granulator and stirred and mixed at 400 rpm for 4 min, and then polyvinyl pyrrolidone solution is added into the mixer and further stirred for 4 min; the shearing is conducted at 800 rpm and continued for 1 min before discharging to make the wet granules; the wet granules are dried and granulated with 1.2 mm mesh, so as to obtain the dry suspension granules III.

### 2) Preparation of Dry Suspension

60.5 mg of Ilaprazole enteric pellet E and 2.0 g of the abovementioned dry suspension granules are mixed and bottled to obtain Ilaprazole enteric dry suspension E-III in the specification of 5 mg.

### 2.4 Preparation of Dry Suspension A-VI

### 1) Preparation of Dry Suspension Granules VI

| Prescription | Dosage |
|---|---|
| Xanthan gum | 25 g |
| Hydroxypropyl methyl cellulose E5 | 8 g |
| Crosslinked sodium carboxymethyl cellulose | 20 g |
| Mannitol | 600 g |
| Sucrose | 250 g |
| Citric acid | 4 g |
| Chitosan | 5 g |
| Purified water | 220 g |

Preparation method: 8 g of hydroxypropyl methyl cellulose E5 is weighed and dissolved in 220 g of purified water, and the prescription dosage of xanthan gum, crosslinked sodium carboxymethyl cellulose, chitosan, mannitol, sucrose, and citric acid are weighed and delivered into HLSH2-6 wet mixing granulator and stirred and mixed at 400 rpm for 4 min, and then hydroxypropyl methyl cellulose E5 solution is added into the mixer and further stirred for 3 min; the shearing is conducted at 1000 rpm and continued for 1 min before discharging to make the wet granules; the wet granules are dried and granulated with 1.2 mm mesh, so as to obtain the dry suspension granules VI.

### 2) Preparation of Dry Suspension

94.5 mg of Ilaprazole enteric pellet A and 1.6 g of the abovementioned dry suspension granules are mixed and bottled to obtain Ilaprazole enteric dry suspension A-VI in the specification of 5 mg.

### 2.5 Preparation of Dry Suspension A-VII

### 1) Preparation of Dry Suspension Granules VII

| Prescription | Dosage |
|---|---|
| Xanthan gum | 30 g |
| Hydroxypropyl methyl cellulose VLV | 18 g |
| Crosslinked sodium carboxymethyl cellulose | 30 g |
| Mannitol | 600 g |
| Sucrose | 260 g |
| Citric acid | 6 g |
| Chitosan | 3 g |
| Purified water | 280 g |

Preparation method: 18 g of hydroxypropyl methyl cellulose VLV is weighed and dissolved in 280 g of purified water, and the prescription dosage of xanthan gum, crosslinked sodium carboxymethyl cellulose, chitosan, mannitol, sucrose, and citric acid are weighed and delivered into HLSH2-6 wet mixing granulator and stirred and mixed at 400 rpm for 4 min, and then hydroxypropyl methyl cellulose VLV solution is added into the mixer and further stirred for 3 min; the shearing is conducted at 800 rpm and continued for 1 min before discharging to make the wet granules; the wet granules are dried and granulated with 1.2 mm mesh, so as to obtain the dry suspension granules VII.

### 2) Preparation of Dry Suspension

94.5 mg of Ilaprazole enteric pellet A and 2.0 g of the abovementioned dry suspension granules are mixed and bottled to obtain Ilaprazole enteric dry suspension A-VII in the specification of 5 mg.

### 2.6 Preparation of Dry Suspension A-VIII

### 1) Preparation of Dry Suspension Granules VIII

| Prescription | Dosage |
|---|---|
| Xanthan gum | 20 g |
| Hydroxypropyl methyl cellulose VLV | 18 g |
| Crosslinked sodium carboxymethyl cellulose | 30 g |
| Mannitol | 600 g |
| Sucrose | 260 g |
| Citric acid | 6 g |
| Chitosan | 40 g |
| Purified water | 280 g |

Preparation method: 18 g of hydroxypropyl methyl cellulose VLV is weighed and dissolved in 280 g of purified water, and the prescription dosage of xanthan gum, crosslinked sodium carboxymethyl cellulose, chitosan, mannitol, sucrose, and citric acid are weighed and delivered into HLSH2-6 wet mixing granulator and stirred and mixed at 400 rpm for 4 min, and then hydroxypropyl methyl cellulose VLV solution is added into the mixer and further stirred for 3 min; the shearing is conducted at 800 rpm and continued for 1 min before discharging to make the wet granules; the wet granules are dried and granulated with 1.2 mm mesh, so as to obtain the dry suspension granules VIII.

### 2) Preparation of Dry Suspension

94.5 mg of Ilaprazole enteric pellet A and 1.8 g of the abovementioned dry suspension granules are mixed and bottled to obtain Ilaprazole enteric dry suspension A-VIII in the specification of 5 mg.

### 2.7 Preparation of Dry Suspension A-IX

### 1) Preparation of Dry Suspension Granules IX

| Prescription | dosage |
|---|---|
| Xanthan gum | 20 g |
| Hydroxypropyl methyl cellulose VLV | 20 g |
| Crosslinked sodium carboxymethyl cellulose | 30 g |
| Mannitol | 660 g |
| Sucrose | 260 g |
| Citric acid | 8 g |
| Chitosan | 60 g |
| Purified water | 300 g |

Preparation method: 20 g of hydroxypropyl methyl cellulose VLV is weighed and dissolved in 300 g of purified water, and the prescription dosage of xanthan gum, crosslinked sodium carboxymethyl cellulose, chitosan, mannitol, sucrose, and citric acid are weighed and delivered into HLSH2-6 wet mixing granulator and stirred and mixed at 400 rpm for 4 min, and then hydroxypropyl methyl cellulose VLV solution is added into the mixer and further stirred for 3 min; the shearing is conducted at 800 rpm and continued for 1 min before discharging to make the wet granules; the wet granules are dried and granulated with 1.2 mm mesh, so as to obtain the dry suspension granules IX.

### 2) Preparation of Dry Suspension

94.5 mg of Ilaprazole enteric pellet A and 1.8 g of the abovementioned dry suspension granules are mixed and bottled to obtain Ilaprazole enteric dry suspension A-IX in the specification of 5 mg.

### 2.8 Preparation of Dry Suspension in Contrast

### 1) Prescription of Contrastive Dry Suspension Granules V (Unit: g)

| Component | Weight (g) |
|---|---|
| Xanthan gum | 20 |
| Chitosan | 1400 |
| CCNA | 75 |
| Aspartame | 15 |

Preparation method: Xanthan gum, chitosan, CCNA and aspartame are mixed uniformly and then added into the dry granulator for granulation. The process parameters are shown as follows: Pinch roller spacing 0.2 mm, feeding speed 30 rpm, pinch roller rotation speed 5 rpm, and granulating rotation speed 10 rpm. After the granulation is completed, they are sieved for granulation with the particle size controlled within 0.5-0.7 mm, so as to obtain the dry suspension granules V.

### 2) Preparation of Dry Suspension

60.5 mg of Ilaprazole enteric pellet E and 2.0 g of the abovementioned dry suspension granules V are mixed and bottled to obtain Ilaprazole enteric dry suspension E-V in the specification of 5 mg.

### Embodiment 3: Performance Measurement of Dry Suspension Granules

### 3.1 Viscosity Measurement of Dry Suspension Granules

### 3.1.1 Dry Suspension Granules I in This Invention

15 mL of water is added into 1.5 g of dry suspension granules I, and the suspension is stirred for 60 s; a calibrated pH meter is used to measure the pH value at 3.9, and then the viscosity of the suspension gel is measured continuously.

### 3.1.2 Dry Suspension Granules II in This Invention

25 ml of water is added into 2.5 g of dry suspension granules II, and the suspension is stirred for 60 s; a calibrated pH meter is used to measure the pH value at 3.8, and then the viscosity of the suspension gel is measured continuously.

### 3.1.3 Dry Suspension Granules III in This Invention

20 mL of water is added into 2.0 g of dry suspension granules III, and the suspension is stirred for 60 s; a calibrated pH meter is used to measure the pH value at 4.0, and then the viscosity of the suspension gel is measured continuously.

### 3.1.4 Dry Suspension Granules VI in This Invention

20 mL of water is added into 2.0 g of dry suspension granules VI, and the suspension is stirred for 60 s; a calibrated pH meter is used to measure the pH value at 4.0, and then the viscosity of the suspension gel is measured continuously.

### 3.1.5 Dry Suspension Granules VII in This Invention

20 ml of water is added into 2.0 g of dry suspension granules VII, and the suspension is stirred for 60 s; a calibrated pH meter is used to measure the pH value at 3.9, and then the viscosity of the suspension gel is measured continuously.

### 3.1.6 Dry Suspension Granules VIII in This Invention

20 ml of water is added into 2.0 g of dry suspension granules VIII, and the suspension is stirred for 60 s; a calibrated pH meter is used to measure the pH value at 3.9, and then the viscosity of the suspension gel is measured continuously.

### 3.1.7 Dry Suspension Granules IX in This Invention

20 ml of water is added into 2.0 g of dry suspension granules IX, and the suspension is stirred for 60 s; a calibrated pH meter is used to measure the pH value at 4.0, and then the viscosity of the suspension gel is measured continuously.

### 3.1.8 Dry Suspension Granules IV in Contrast

As a sample of prior art, NEXIUM® (ORAL SUSPENSION, in the specification of 40 mg) from AstraZeneca is used; the enteric pellets containing Esomeprazole magnesium are removed from the granules of the product, and 15 mL of purified water is added into the residual powder, and the suspension is stirred for 60 s, and then the viscosity of the suspension gel is measured continuously.

### 3.1.9 Dry Suspension Granules V in Contrast

20 mL of water is added into 2.0 g of dry suspension granules V in contrast, and the suspension is stirred for 60 s; a calibrated pH meter is used to measure the pH value at 8.0, and then the viscosity of the suspension gel is measured continuously.

Equipment: BROOKFIELD DV2T viscometer, small sample adapter SSA, and MVIY flag-shaped impeller blade rotor.

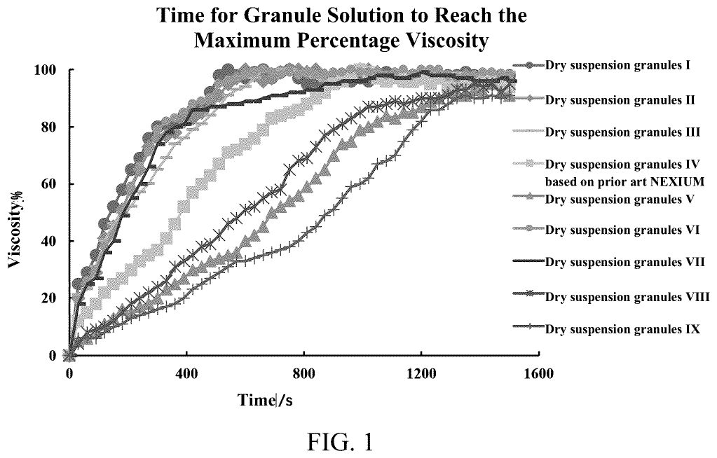

See Table 2 and FIG. 1 below for the viscosity measurement results of the abovementioned dry suspension granules:

TABLE 2
Viscosity Measurement Results of Dry Suspension Granules
Time taken to reach % of maximum viscosity

| | Percentage in maximum viscosity | | | |
|---|---|---|---|---|
| | >75% | | >90% | |
| Dry suspension | Average = | 5.4 min | Average = | 8.8 min |
| granules I in | Minimum = | 4.6 min | Minimum = | 7.5 min |
| this invention | Maximum = | 8.3 min | Maximum = | 11.6 min |
| (n = 6) | | | | |
| Dry suspension | Average = | 5.6 min | Average = | 10.5 min |
| granules II in | Minimum = | 5.0 min | Minimum = | 7.2 min |
| this invention | Maximum = | 9.7 min | Maximum = | 12.3 min |
| (n = 6) | | | | |
| Dry suspension | Average = | 6.5 min | Average = | 9.4 min |
| granules III in | Minimum = | 5.1 min | Minimum = | 8.3 min |
| this invention | Maximum = | 8.8 min | Maximum = | 13.8 min |
| (n = 6) | | | | |
| Dry suspension | Average = | 7.2 min | Average = | 8.9 min |
| granules VI in | Minimum = | 6.5 min | Minimum = | 7.9 min |
| this invention | Maximum = | 8.9 min | Maximum = | 9.6 min |
| (n = 6) | | | | |
| Dry suspension | Average = | 7.9 min | Average = | 8.6 min |
| granules VII in | Minimum = | 6.8 min | Minimum = | 6.3 min |
| this invention | Maximum = | 9.8 min | Maximum = | 10.5 min |
| (n = 6) | | | | |
| Dry suspension | Average = | 18.9 min | Average = | 19.5 min |
| granules VIII in | Minimum = | 17.2 min | Minimum = | 16.9 min |
| this invention | Maximum = | 22.3 min | Maximum = | 24.2 min |
| (n = 6) | | | | |
| Dry suspension | Average = | 19.8 min | Average = | 22.5 min |
| granules IX in | Minimum = | 14.6 min | Minimum = | 21.5 min |
| this invention | Maximum = | 22.1 min | Maximum = | 24.6 min |
| (n = 6) | | | | |
| Dry suspension | Average = | 9.7 min | Average = | 13.5 min |
| granules IV in | Minimum = | 7.2 min | Minimum = | 9.1 min |
| contrast based | Maximum = | 11.5 min | Maximum = | 20.3 min |
| on prior art | | | | |
| NEXIUM ® | | | | |
| (n = 6) | | | | |
| Dry suspension | Average = | 19.7 min | Average = | 23.5 min |
| granules V in | Minimum = | 15.2 min | Minimum = | 19.1 min |
| contract (n = 6) | Maximum = | 21.5 min | Maximum = | 30.3 min |

It can be seen from Table 2 and FIG. 1 that the dry suspension granules I-III and VI-VIII in this invention can reach the maximum viscosity faster than the dry suspension granules IV based on prior art; due to the large dosage of chitosan, it will take a longer time obviously for dry suspension granules V in contrast to reach the maximum viscosity after water is added; furthermore, the dry suspension granules V in contrast will become alkaline after added into water; as a result, the enteric coating of Ilaprazole enteric pellets will be dissolved.

### 3.2 Test on the Time Required for Ilaprazole Pellets in the Dry Suspension to Maintain Suspension

(1) 15 mL of water is added into the dry suspension A-I in the embodiment 8.1, the suspension is stirred at constant speed for 30 s; at this moment, the timer is started to observe the time taken for all the enteric pellets to suspend.
(2) 25 mL of water is added into the dry suspension D-II in the embodiment 8.2, the suspension is stirred at constant speed for 30 s; at this moment, the timer is started to observe the time taken for all the enteric pellets to suspend.
(3) 20 mL of water is added into the dry suspension E-III in the embodiment 8.3, the suspension is stirred at constant speed for 30 s; at this moment, the timer is started to observe the time taken for all the enteric pellets to suspend.
(4) 15 mL of purified water is added into NEXIUM® (ORAL SUSPENSION, in the specification of 40 mg) from AstraZeneca, the suspension is stirred at constant speed for 30 s; at this moment, the timer is started to observe the time taken for all the enteric pellets to suspend.
(5) 20 ml of water is added into the dry suspension E-V in contrast in the embodiment 8.4, the suspension is stirred at constant speed for 30 s; at this moment, the timer is started to observe the time taken for all the enteric pellets to suspend.

See Table 20 below for the results of the abovementioned tests:

TABLE 3
Test Results on the Time Required for Ilaprazole
Pellets in Dry Suspension to Maintain Suspension

| | Time for pellets to |
|---|---|
| Sample | maintain suspension |
| Dry suspension A-I in this invention | 38 s |
| Dry suspension D-II in this invention | 45 s |
| Dry suspension E-III in this invention | 33 s |
| Dry suspension IV based on prior art | 119 s |
| NEXIUM ® | |
| Dry suspension E-V in contrast | 735 s |

It can be seen from Table 3 that dry suspensions A-I, D-II and E-III in this invention can form suspension gel in a shorter time than those based on prior art; the time required for the dry suspended pellets in dry suspension E-V in contrast to maintain suspension is 735 s, and the suspension velocity is affected due to the too high gel strength.

### 3.3 Suspension-Sedimentation Time of Pellets in Suspension Gel

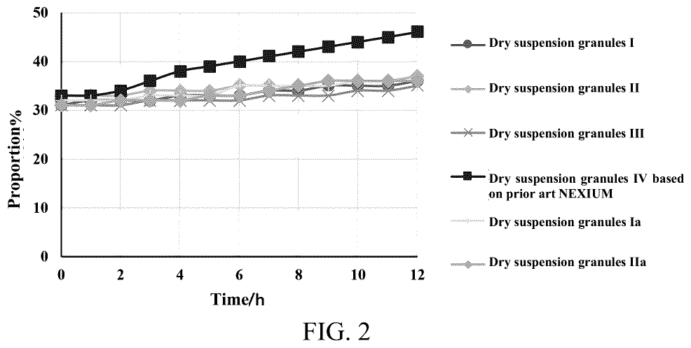

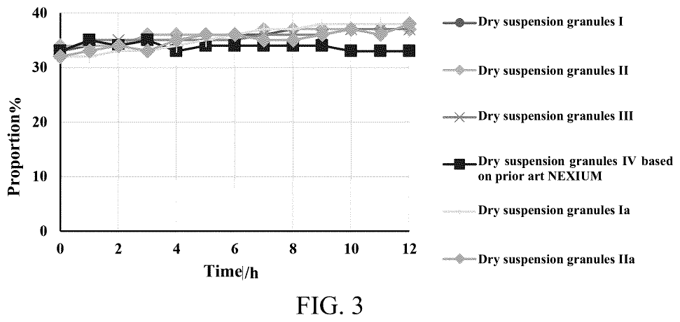

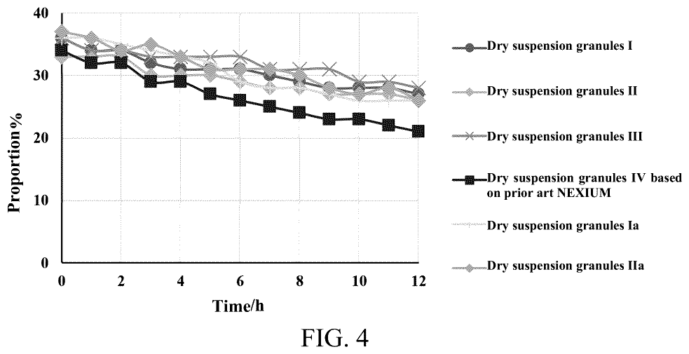

(1) 100 Ilaprazole enteric pellets A and 15 mL of water are added into 1.5 g of dry suspension granules I in this invention, and the suspension is stirred for 150 s until the enteric pellets are suspending fully; and then the suspension is poured into a 15 mL volumetric cylinder, the number of enteric pellets within the measuring range of 0-5 mL, 5-10 mL, and 10-15 mL is counted respectively, and the calculation is made once every 1 h within a duration of 12 h. Ten groups of tests are conducted simultaneously. The average proportion of the pellets within the measuring range of 0-5 mL, 5-10 mL, and 10-15 mL is calculated (see the curve of dry suspension granules I in FIGS. 2-4).
(2) 100 Ilaprazole enteric pellets D and 15 mL of water are added into 1.5 g of dry suspension granules II in this invention, and the suspension is stirred for 150 s until the enteric pellets are suspending fully; and then the suspension is poured into a 15 mL volumetric cylinder, the number of enteric pellets within the measuring range of 0-5 mL, 5-10 mL, and 10-15 mL is counted respectively, and the calculation is made once every 1 h within a duration of 12 h. Ten groups of tests are conducted simultaneously. The average proportion of the pellets within the measuring range of 0-5 mL, 5-10 mL, and 10-15 mL is calculated (see the curve of dry suspension granules II in FIGS. 2-4).
(3) 100 Ilaprazole enteric pellets E and 15 mL of water are added into 1.5 g of dry suspension granules III in this invention, and the suspension is stirred for 150 s until the pellets are suspending fully; and then the suspension is poured into a 15 mL volumetric cylinder, the number of enteric pellets within the measuring range of 0-5 mL, 5-1 0mL, and 10-15 mL is counted respectively, and the calculation is made once every 1 h within a duration of 12 h. Ten groups of tests are conducted simultaneously. The average proportion of the pellets within the measuring range of 0-5 mL, 5-10 mL, and 10-15 mL is calculated (see the curve of dry suspension granules III in FIGS. 2-4).
(4) The dry suspension granules IV are from a product based on prior art, i.e. NEXIUM® (ORAL SUSPENSION, in the specification of 40 mg) from AstraZeneca; the enteric pellets containing Esomeprazole magnesium are completely removed from the dry suspension granules of the product, and 100 Esomeprazole magnesium enteric pellets and 15 mL of water are added into such granules, and the suspension is stirred for 150 s until the pellets are suspending fully; and then the suspension is poured into a 15 mL volumetric cylinder, the number of enteric pellets within the measuring range of 0-5 mL, 5-10 mL, and 10-15 mL is counted respectively, and the calculation is made once every 1 h within a duration of 12 h. Ten groups of tests are conducted simultaneously. The average proportion of the pellets within the measuring range of 0-5 mL, 5-10 mL, and 10-15 mL is calculated (see the curve of dry suspension granules IV based on prior art NEXIUM in FIGS. 2-4).
(5) 100 Esomeprazole magnesium enteric pellets of NEXIUM® (ORAL SUSPENSION) from AstraZeneca and 15 mL of water are added into 1.5 g of dry suspension granules I in this invention, and the suspension is stirred for 150 s until the enteric pellets are suspending fully; and then the suspension is poured into a 15 mL volumetric cylinder, the number of enteric pellets within the measuring range of 0-5 mL, 5-10 mL, and 10-15 mL is counted respectively, and the calculation is made once every 1 h within a duration of 12 h. Ten groups of tests are conducted simultaneously. The average proportion of the pellets within the measuring range of 0-5 mL, 5-10 mL, and 10-15 mL is calculated (see the curve of dry suspension granules la in FIGS. 2-4).
(6) 100 Celphere CP-507 microcrystalline cellulose blank pellet cores (particle size ranging from 500 μm to 700 μm) from Asahi Kasei and 15 mL of water are added into 1.5 g of dry suspension granules II in this invention, and the suspension is stirred for 150 s until the enteric pellets are suspending fully; and then the suspension is poured into a 15 mL volumetric cylinder, the number of enteric pellets within the measuring range of 0-5 mL, 5-10 mL, and 10-15 mL is counted respectively, and the calculation is made once every 1 h within a duration of 12 h. Ten groups of tests are conducted simultaneously. The average proportion of the pellets within the measuring range of 0-5 mL, 5-10 mL, and 10-15 mL is calculated (see the curve of dry suspension granules IIa in FIGS. 2-4).

The results of the above tests are shown in FIG. 2-FIG. 4. The results show that: Within the measuring range of 0-5 ml, the proportion of the enteric pellets in the suspension formed by the dry suspension granules IV based on prior art is higher than that in the suspension formed by the dry suspension granules I, II or III based on this invention; furthermore, as the time goes by, the proportion of the enteric pellets in the suspension formed by the dry suspension granules IV increases significantly. In contrast, within the measuring range of 10-15 ml, the proportion of the enteric pellets in the suspension formed by the dry suspension granules IV is lower than that in the suspension formed by the dry suspension granules I, II or III; furthermore, as the time goes by, the proportion of the enteric pellets in the suspension formed by the dry suspension granules IV becomes lower and lower. These facts indicate that with the suspension/suspension gel prepared with the dry suspension granules based on this invention, the enteric pellets can suspend for a longer time and are less likely to sediment.

### 3.4 Measurement of Acid Resistance of Ilaprazole Enteric Pellets in Different Dry Suspensions

The acid resistance of Ilaprazole enteric pellets in the dry suspension based on this invention and the dry suspension in contrast is measured by reference to the following measurement method. Please see the results in Table 4 below:

Measurement method: The dry suspension containing enteric pellets is taken with 0.1 mol/L hydrochloric acid solution (9.0 mL of concentrated hydrochloric acid is added with water to the volume of 1000 mL) as the dissolution medium, and the rotation speed of 100 rpm. After 120 min, the dissolution cup is taken out and the solution is filtered with suction filter, and the residual pellets are collected into a 50 mL volumetric flask; 20 mL of 0.05 mol/L sodium hydroxide solution is added, and the solution is poured into an oscillator for agitation (250 rpm) for 20 min; 30 mL of methanol is added for ultrasound processing for 10 min, and then water is added for dilution to the scale, and the supernatant is taken after shaking well and centrifuging. The content of residual drug in the pellets is measured, i.e. the acid resistance of the sample.

TABLE 4
Measurement Results of Acid Resistance of Ilaprazole
Enteric Pellets in Different Dry Suspensions

| | Dry | Dry | Dry | Dry |
|---|---|---|---|---|
| | Suspension | Suspension | Suspension | Suspension |
| Duration | A-I | D-II | E-III | E-V |
| 0 day | 97.5% | 95.5% | 96.4% | 95.6% |
| 1 month | 95.6% | 96.1% | 95.8% | 96.8% |
| 3 months | 96.4% | 95.7% | 96.5% | 95.4% |
| 6 months | 94.5% | 96.0% | 97.5% | 94.8% |

### 3.5 Measurement of Dissolution Rate of Ilaprazole Enteric Pellets in Different Dry Suspensions

The dissolution rate of Ilaprazole enteric pellets in the dry suspension based on this invention and the dry suspension in contract is measured by reference to the measurement method for dissolution rate and release rate (see the first method under Method 2 in the General Rule 0931 of Chinese Pharmacopoeia 2015 Edition Volume IV for the general method). The results of measurement are shown in Table 5 below:

### Measurement Method:

The sample to be tested is taken, with 300 mL of 0.1 mol/L hydrochloric acid solution (9.0 mL of hydrochloric acid is added with water to the volume of 1000 mL) as the dissolution medium, and the rotation speed of 100 rpm, and the operations are carried out according to the method for 120 min; at this moment, 700 mL of 0.086 mol/L disodium hydrogen phosphate solution preheated to 37±0.5° C. (30.8 g of disodium hydrogen phosphate and 7 g of Tween 80 are taken, and water is added to the volume of 1000 mL) is added into the dissolution cups, and then mixed uniformly while the rotation speed remains unchanged. The operations are carried out according to the method for 45 min; at this moment, the sample is taken.

Test sample solution: An appropriate amount of the dissolved solution is taken and filtered, 5 ml of subsequent filtrate is measured accurately, and 1 mL of 0.15 mol/L sodium hydroxide solution is added accurately and immediately, and then the solution is shaken well and filtered; the subsequent filtrate is taken as the test sample solution.

Reference substance solution: About 10 mg of Ilaprazole reference substance is weighed accurately and put into a 20 mL volumetric flask, and appropriate amount of acetonitrile is added to dissolve it, and the solution is diluted to the scale with acetonitrile and shaken well; 1 mL of the solution is weighed accurately and put into a 100 mL volumetric flask, and then diluted to the scale with phosphate buffer solution (pH 6.8) (700 mL of 0.086 mol/L disodium hydrogen phosphate solution and 300 mL of 0.1 mol/L hydrochloric acid solution are mixed uniformly to obtain such phosphate buffer solution) and shaken well; 5 mL of such solution is weighed accurately, and 1 mL of 0.05 mol/L sodium hydroxide solution is added accurately and immediately, and then the solution is shaken well and filtered; the subsequent filtrate is taken as the reference substance solution.

TABLE 5
Measurement Results of Dissolution Rate of Ilaprazole
Enteric Pellets in Different Dry Suspensions

| | Dry | Dry | Dry | Dry |
|---|---|---|---|---|
| | Suspension | Suspension | Suspension | Suspension |
| T/min | A-I | D-II | E-III | E-V |
| 0 | 0% | 0% | 0% | 0% |
| 10 | 35.5% | 38.7% | 34.5% | 15.6% |
| 15 | 58.4% | 59.6% | 46.8% | 20.5% |
| 20 | 79.5% | 83.5% | 59.4% | 29.5% |
| 30 | 86.5% | 96.5% | 78.5% | 36.4% |
| 45 | 92.5% | 95.4% | 91.2% | 45.9% |
| 60 | 93.2% | 94.6% | 92.5% | 60.5% |

In summary, dry suspension E-V is a kind of suspension preparation made with the prescription in contrast. Despite its acid resistance is equivalent to that of the suspension preparation based on this invention, its dissolution rate is slowed down obviously due to the higher dosage of chitosan, resulting in the too high strength of gel and the impact on the dissolution rate of the pellets.

As seen from the above results, by using the combination of anionic gel and cationic polymer (especially chitosan and its derivatives) in the Ilaprazole dry suspension based on this invention, the suspension can reach a stable viscosity level quickly, and the suspension gel so prepared enables the enteric pellets to suspend stably for a longer period of time. Furthermore, the dissolution rate of the acid-unstable proton pump inhibitors, especially Ilaprazole, can be increased with such suspension gel.

1. The dry suspension granules containing anionic gel and cationic polymer are characterized by the fact that the weight ratio between the said anionic gel and the said cationic polymer is (0.5-50):1, preferably (0.8-20):1, more preferably (0.9-10):1, and most preferably (1-3):1.

2. The dry suspension granules as set forth in claim 1 are characterized by the fact that the said anionic gel is sourced from one, two or more of Arabic gum, gelatin, alginate (e.g. sodium alginate), pectin, xanthan gum, gellan gum, locust bean gum, guar gum, agar, carrageenan, tamarind gum, konjac gum, cassia bean gum, tragacanth gum, and karaya gum.

3. The dry suspension granules as set forth in claim 1 characterized by the fact that the said cationic polymer is sourced from chitosan or its derivatives; the weight percentage of the said cationic polymer in dry suspension granules should be 0.5-5%, preferably 0.6-4%, more preferably 0.8-3.5%, and most preferably 0.9-3.0%.

4. The dry suspension granules as set forth in claim 1 are characterized by the fact that the dry suspension granules based on this invention also include one, two or more of adhesive, disintegrating agent, diluent, and pH regulator; preferably, the said adhesive is sourced from one, two or more of polyvinyl pyrrolidone, hydroxypropyl methyl cellulose, hydroxypropyl cellulose, methyl cellulose, polyvinyl alcohol, and starch; preferably, the said disintegrating agent is sourced from one, two or more of sodium carboxymethyl cellulose, calcium carboxymethyl cellulose, crosslinked sodium carboxymethyl cellulose, crosslinked sodium carboxymethyl starch, low-substituted hydroxypropyl cellulose, crosslinked polyvinyl pyrrolidone, microcrystalline cellulose, and pre-gelatinized starch; preferably, the said diluent is sourced from one, two or more of xylitol, mannitol, sucrose, glucose, sorbitol, maltitol, and fructose; preferably, the said pH regulator is sourced from one, two or more of organic or inorganic acids, preferable tartaric acid, citric acid, oxalic acid, succinic acid, fumaric acid, ascorbic acid, malic acid, glutamic acid, and caffeic acid.

5. The dry suspension granules as set forth in claim 1 4 are characterized by the fact that the pH value of the suspension gel formed by the said dry suspension granules in the water-based medium ranges from 2.5 to 7.0, and preferably from 3.0 to 5.0; and/or the amount of water-bearing dispersion medium added into the dry suspension granules based on this invention is 2 to 50 times of the weight of the dry suspension granules when preparing the suspension gel.

6. The method for preparing the dry suspension granules as set forth in claim 1 includes the following steps:
(1) Mix the anionic gel and the cationic polymer as well as other optional excipients;
(2) Prepare separately the adhesive solution;
(3) Add the adhesive solution obtained in Step (2) into the mixture obtained in Step (1), to prepare the wetting mixture;
(4) Granulate the wetting mixture obtained in Step (3) to obtain the dry suspension granules based on this invention.
Preferably, the said other excipients include diluent, pH regulator, disintegrating agent, etc.
Preferably, water and/or ethanol should be used as solvent in Step (2).

7. The method for preparing the dry suspension granules as set forth in claim 1 includes the following steps:
(1) Mix the anionic gel and the cationic polymer as well as other optional excipients;
(2) Provide water and/or ethanol as the wetting agent;
(3) Add the wetting agent provided in Step (2) into the mixture obtained in Step (1), to prepare the wetting mixture;
(4) Granulate the wetting mixture obtained in Step (3), to obtain the dry suspension granules based on this invention.
Preferably, the said other excipients include diluent, pH regulator, disintegrating agent, and/or adhesive, etc.
Preferably, in the method for preparing dry suspension granules based on this invention, following Step (4) there is Step (5): Make the dry suspension granules based on this invention by drying.
Preferably, in the method for preparing the dry suspension granules based on this invention, following Step (5) there is Step (6): Granulate the dry suspension granules based on this invention in the form of pellet.

8. The pharmaceutical composition, in particular the dry suspension, contains the dry suspension granules as set forth in claim 1; preferably, the said pharmaceutical composition contains the enteric pellets, in particular the enteric pellets containing Ilaprazole; more preferably, the mass ratio between the dosage of Ilaprazole in the said enteric pellets and the dosage of the dry suspension granules is 1:200-1000.

9. The pharmaceutical composition as set forth in claim 8, preferably the dry suspension, is characterized by the fact that the duration in which the suspension gel obtained by adding the dispersion medium into the said pharmaceutical composition reaches 75% of the maximum viscosity is less than 8 min, preferably less than 7 min; preferably, the duration in which the suspension gel obtained reaches 90% of the maximum viscosity is less than 12 min, preferably less than 11 min; preferably, the duration in which the suspension gel is formed by adding the water-bearing medium into the said pharmaceutical composition is less than 120 s, preferably less than 90 s, more preferably less than 70 s, and most preferably less than 50 s; preferably, in the case of including enteric pellets that contain Ilaprazole, the release rate of Ilaprazole should not exceed 10% within 1 h after adding the said pharmaceutical composition into the dispersion medium to form the solution with pH 1.2, and/or the release rate of Ilaprazole should not be less than 70% within 45 min after forming the solution with pH 6.8.

10. The method for treatment and/or prevention of gastrointestinal diseases, comprising administering effective dose of the dry suspension as set forth in claim 1 for treatment and/or prevention to the patients who need such treatment and/or prevention
the said gastrointestinal diseases include heartburn, inflammatory bowel disease, Crohn's disease, irritable bowel syndrome, ulcerative colitis, peptic ulcer, stress ulcer, bleeding peptic ulcer, duodenal ulcer and recurrent duodenal ulcer, gastric ulcer associated with NSAID, adult active benign gastric ulcer, infective enteritis, colitis, hyperacidity, dyspepsia, gastroparesis, Zollinger-Ellison syndrome, gastroesophageal reflux disease (GERD), diseases related to helicobacter pylori or eradication of helicobacter pylori, erosive esophagitis and short bowel syndrome at all levels, or any combination of the above diseases.
